(19) United States
(12) Patent Application Publication
(10) Pub. No.: US 2024/0180675 A1
(43) Pub. Date: Jun. 6, 2024

(54) FUNCTIONAL IMPRESSION FOR DENTAL PROSTHESES

(71) Applicant: Ivoclar Vivadent AG, Schaan (LI)

(72) Inventor: Theresa Sujata Maria Senti, Triesenberg (LI)

(21) Appl. No.: 18/525,374

(22) Filed: Nov. 30, 2023

(30) Foreign Application Priority Data
Dec. 1, 2022 (EP) 22210843.3

(51) Int. Cl. A61C 13/00; A61B 1/00; A61B 1/24; A61C 9/00

(52) U.S. Cl. CPC A61C 13/0004; A61B 1/00172; A61B 1/24; A61C 9/0053

(57) ABSTRACT

A method of determining the three-dimensional shape of a functional surface of a dental prosthesis, comprising the steps of detecting (S101) an intraoral space to generate a three-dimensional actual data set; and inputting (S102) the three-dimensional data set into a learned algorithm to generate a three-dimensional nominal data set for a functional surface of the dental prosthesis.

### CROSS-REFERENCE TO RELATED APPLICATIONS

This application claims priority to European Patent Application No. 22210843.3 filed on Dec. 1, 2022, the disclosure of which is incorporated herein by reference in its entirety.

### TECHNICAL FIELD

The present invention relates to a method of determining the three-dimensional shape of a functional surface of a dental prosthesis and a device for determining the three-dimensional shape of a functional surface of a dental prosthesis.

### BACKGROUND

Currently, dental prostheses are produced using several impression procedures and then converted into a dental prosthesis by a dental technician. Presently, the anatomical impression is made using a standard tray and impression material. This impression is then sent to a dental laboratory. There, the individual tray for the functional impression is produced on the basis of this impression.

This tray is then sent back to the dentist, who can now take the functional impression for the dental prosthesis. In taking the functional impression, the esthetic lines and the occlusal plane are recorded in addition to the functional margin. By means of the thickness of the impression in the anterior region, the lip support (soft tissue support/aesthetics) is also defined. In addition, the tooth color, tooth shape or tooth type are also determined. The impression and this additional information are finally passed on to the dental technician, who now begins to produce the total prosthesis. Up to now, this procedure has been analogous and there is no alternative to the conventional functional impression with a functional tray, especially for the edentulous jaw.

### SUMMARY

It is the technical task of the present invention to simplify and accelerate the design of functional surfaces of a dental prosthesis.

This technical task is solved by subject matter according to the independent claims. Technically advantageous embodiments are the subject of the dependent claims, the description and the drawings.

According to a first aspect, the technical task is solved by a method of determining the three-dimensional shape of a functional surface of a dental prosthesis, comprising the steps of scanning an intraoral space to generate a three-dimensional actual data set; and inputting the three-dimensional data set into a learned algorithm to generate a three-dimensional nominal data set for a functional surface of the dental prosthesis. Additionally, selected landmarks as well as supplemental data sets may be input, such as two-dimensional images. The dental prosthesis can be a full or partial prosthesis. The method can be used to determine a line, such as a functional margin, or corresponding marks on the three-dimensional data set. In addition, a faster, simpler, and more efficient production of a dental prosthesis is achieved. US 20210321872 is directed to intraoral scanners and is hereby incorporated by reference in its entirety.

In a technically advantageous embodiment of the method, the learned algorithm comprises an artificial neural network. The artificial neural network can be a deep neural network (DNN) or a transformer network. This provides the technical advantage that the nominal data set can be calculated efficiently. US 20190282344 is directed to dental CAD automation using Deep Learning and is hereby incorporated by reference in its entirety.

U.S. Pat. Nos. 11,694,418, 11,694,418, 11,735,306 and 11,783,539 are directed to machine learning architecture systems and methods and are hereby incorporated by reference in their entirety.

In another technically advantageous embodiment of the method, the artificial neural network comprises multiple layers.

In another technically advantageous embodiment of the method, the artificial neural network comprises a convolution layer, a pooling layer, and/or a dense layer.

In another technically advantageous embodiment of the method, the intraoral space is scanned with a scanner. In general, the intraoral space can be scanned by any system to gather data that can be directly or indirectly converted into a 3D model. This also provides the technical advantage that the three-dimensional actual data set can be obtained more quickly than with an impression scan.

In another technically advantageous embodiment of the method, the functional surface is a contact surface of the dental prosthesis to the soft tissue. This also provides the technical advantage that the dental prosthesis can be adapted to the soft tissue.

In a further technically advantageous embodiment of the method, the three-dimensional actual data set comprises additional data on soft tissue elasticity. This also provides the technical advantage that the spatial shape of the dental prosthesis can be adapted in such a way that wearing comfort is improved.

In another technically advantageous embodiment of the method, the three-dimensional actual data set comprises additional spatial data on an upper jaw, a lower jaw and/or a face. This also provides the technical advantage of improving the fit of the dental prosthesis in the intraoral space. In addition, together with the information about a face, esthetics can be better considered or planned.

In another technically advantageous embodiment of the method, the learned algorithm calculates a functional margin of the dental prosthesis. This also provides the technical advantage that the dental prosthesis can be adapted to the functional movements of the soft tissue.

In another technically advantageous embodiment of the method, the learned algorithm calculates a tooth set-up or tooth set. This also provides the technical advantage of accelerating the design of the dental prosthesis.

In another technically advantageous embodiment of the method, the dental prosthesis is produced with the generated functional surface. This also provides the technical advantage that the dental prosthesis can be produced automatically with the correct functional surface.

According to a second aspect, the technical task is solved by a system for determining a three-dimensional shape of a functional surface of a dental prosthesis; comprising an intraoral scanner for scanning an intraoral space to generate a three-dimensional actual data set; and a learned algorithm for inputting the three-dimensional data set and outputting a three-dimensional nominal data set for a functional surface of the dental prosthesis. The system provides the same technical advantages as the method according to the first aspect.

In a technically advantageous embodiment of the method, the learned algorithm comprises an artificial neural network. This provides the technical advantage that the nominal data set can be calculated efficiently.

In another technically advantageous embodiment of the system, the system comprises a production device for producing the dental prosthesis with the generated functional surface. This also provides the technical advantage that the dental prosthesis can be produced automatically with the correct functional surface.

The system may include computer (s)/devices and server computer (s) to provide processing, storage, and input/output devices executing application programs and the like. The computer (s)/devices can also be linked through communications network to other computing devices. The communications network can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

I/O device interfaces for connecting various input and output devices include, but are not limited to e.g., keyboard, mouse, displays, printers, speakers, etc. A memory provides volatile storage for computer software instructions and data used to implement an embodiment of the present invention. Disk storage provides non-volatile storage for computer software instructions and data used to implement an embodiment of the present invention. A central processor unit can be used to provide for the execution of computer instructions.

In one embodiment, the processor routines and data are a computer program product, including a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network (s)). Such carrier medium or signals may be employed to provide at least a portion of the software instructions for the present invention routines/program.

According to a third aspect, the technical task is solved by a computer program comprising instructions which, when executed by a system for determining a three-dimensional shape of a functional surface of a dental prosthesis, cause the method according to the first aspect to be executed. The computer program provides the same technical advantages as the method according to the first aspect. The computer program product has program code which is stored on a non-transitory machine-readable medium, the machine-readable medium including computer instructions executable by a processor, which computer instructions cause the processor to perform the method as set forth above.

### BRIEF DESCRIPTION OF THE DRAWINGS

Exemplary embodiments of the invention are shown in the drawings and are described in more detail below, in which:

FIG. 1 shows a schematic view of a system for determining a three-dimensional shape of a functional surface of a dental prosthesis; and

FIG. 2 shows a block diagram of the method for determining the three-dimensional shape of a functional surface of a dental prosthesis.

### DETAILED DESCRIPTION

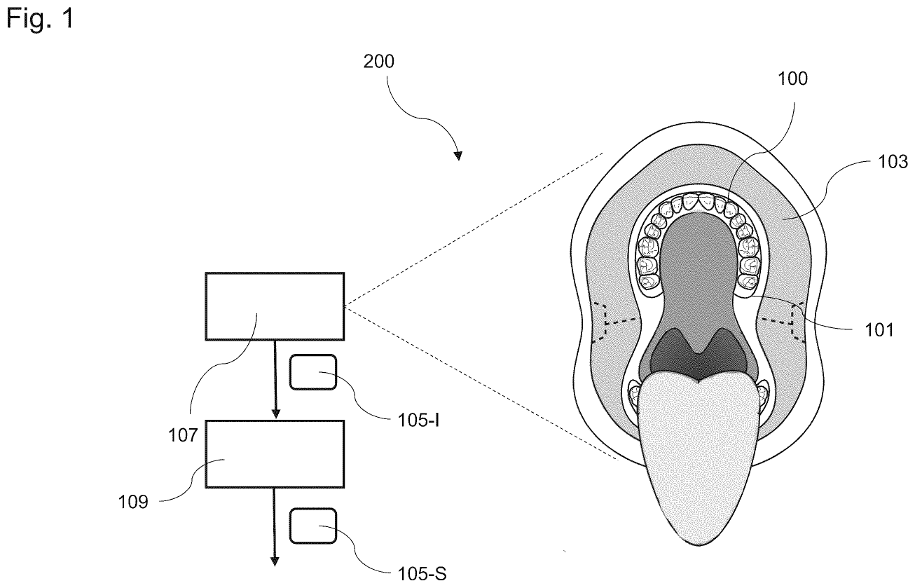

FIG. 1 shows a schematic view of a system 200 for determining a three-dimensional shape of a functional surface 101 of a dental prosthesis 100.

The functional surface 101 comprises, for example, the entire three-dimensional design of the dental prosthesis 100. Anatomical, functional and aesthetic aspects can be taken into account. For example, the functional surface 101 comprises a contact surface of the dental prosthesis 100 to the soft tissue in the oral cavity. For example, the functional surface 101 comprises an occlusal surface or tooth set-up of one or more teeth. The way the dental prosthesis 100 is configured, such as by the shape, size, and/or angle of the teeth, has a great influence on the appearance of the person wearing the dental prosthesis 100.

The system 200 comprises an intraoral scanner 107 for scanning an intraoral space 103 to obtain a three-dimensional actual data set 105-I of the intraoral space 103. The actual data set 105-I indicates the spatial shape of the soft tissue in the mouth. In addition, upper and lower jaw or face can be scanned to obtain additional three-dimensional data. From the additional scans, a relation between the scans of the upper jaw, lower jaw and face can be obtained. The individual scans (intraoral and extraoral) are precisely aligned with each other in the correct position. This is a technical advantage over current systems, which use a reference object for best-fit alignment.

In addition, information can be gathered about the spatial position between the scans performed in different areas. Furthermore, various facial expressions can be detected, such as when laughing or speaking. The three-dimensional information can be gathered from different angles. Additional data, such as a functional margin, a centerline, or a Camper's level, can be specified in the intraoral scan.

In addition, information about the facial tissue and the elasticity of the soft tissue can be gathered, such as by magnetic resonance imaging (MRI) or by simultaneously detecting, scanning or filming the face and oral cavity while the patient moves the face. This can be done by an intraoral scanner 107, which can move the tissue in a controlled manner via a balloon and can directly determine the tissue resistance.

The system comprises a learned algorithm 109 for inputting the three-dimensional actual data set 105-I and outputting a three-dimensional nominal data set 105-S for a functional surface 101 of the dental prosthesis 100. The learned algorithm 109 is stored in a digital memory, for example, and executed by a processor. The digital memory may store, for example, other data used in executing the algorithm.

The learned algorithm 109 automatically calculates the spatial shape of the functional surface 101 or the functional margin from the spatial shape of the intraoral space 103. To do this, the algorithm 109 is previously learned with a plurality of training data that combine a three-dimensional shape of the intraoral space and an associated spatial shape of a functional surface 101. For example, the learned algorithm may comprise an artificial neural network. The artificial neural network is based on interconnecting many neurons, each of which is interconnected by means of a weighting. The neural network comprises multiple layers that perform various calculations, such as a convolution layer, a pooling layer, or a dense layer. The neural network can also be based on a GAN (Generative Adversarial Networks) architecture.

Three-dimensional data sets of an intraoral space are used as training data for the neural network, each of which is assigned in advance a corresponding three-dimensional data set for the functional surface 101. In addition, further information can be used, such as a facial scan. The three-dimensional data set of the intraoral space for the training data can, for example, be obtained from an impression of a edentulous jaw with a high degree of precision. This data set is then assigned a functional surface 101, which has, for example, been determined by a conventional method.

The weightings between the artificial neurons of the artificial neural network are adjusted in the training phase such that when a data set for the intraoral space is input, the respective data set for the functional surface 101 is output. If, after training of the neural network, a three-dimensional actual data set 105-I of an intraoral space 103 from a performed scan is then input, a functional surface 101 of the dental prosthesis 100 is automatically output by the neural network. Thereby, a nominal data set for a functional surface 101, which is similar to the functional surface 101 of corresponding training data, is output for the inputted actual data set 105-I.

Moreover, the three-dimensional data sets of the intraoral space 103 for training the neural network may comprise predetermined information, such as:
information about a jaw relation including an intervestibular distance, a physiological rest position, or a centric relation;
information about an occlusal plane, for example a Camper's plane or a Frankfurt horizontal;
skull-related information, for example a skull to upper jaw ratio, a facebow, or a mean-value Bonwill Triangle;
information about a functional margin or a frenulum passage, for example an A-line, an outer and inner bead seal or a muco-gingival line;
information about a fullness of the lips;
information about a bipupillary line;
information about an aesthetic line, such as a centerline, a cuspid line, a smile line, or a lip closure line;
information about a Gothic arc, such as arrow point tracing or needlepoint tracing;
information about tooth color and shape;
information about a width of the nose as a starting point for the size of the teeth;
information about a papilla incisiva or palanine raphe; and/or
information about a Gothic arc (arrow point or needlepoint tracing)

The information can be determined and made available directly or indirectly. It can be used as input to generate a suggestion of what the prosthesis might look like. In addition, this information can be used as a basis to train the neural network. This can be used to advise the patient and discuss different variations of tooth shapes and sizes. For this purpose, the proposed prosthesis is displayed in the mouth together with the data gathered from the face.

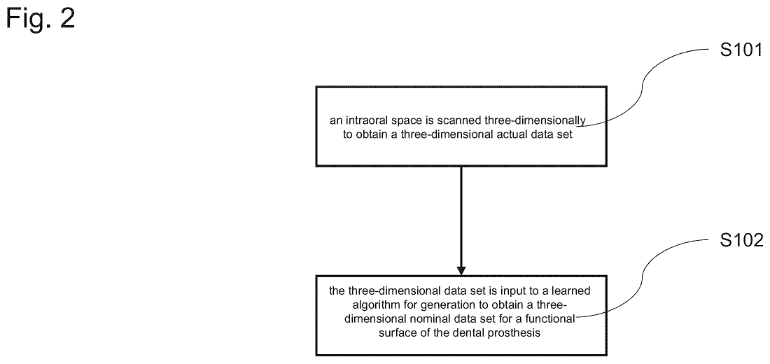

FIG. 2 shows a method of determining the three-dimensional shape of a functional surface 101 of a dental prosthesis 100. In step S101, the intraoral space 103 is scanned three-dimensionally to obtain a three-dimensional actual data set 105-I. This three-dimensional actual data set 105-I represents the spatial shape of the soft tissue in the intraoral space. Direct gathering of the soft tissue can be performed in the intraoral space without complex equipment and without radiation exposure using an intraoral scanner 107. During scanning, for example, a scan of the upper jaw or lower jaw including soft tissue can also be performed. In addition, a scan can be performed with videos or photos of the face or head.

In step S102, the three-dimensional data set 105-I is input to a learned algorithm for generation to obtain a three-dimensional nominal data set 105-S for a functional surface 101 of the dental prosthesis 100.

Based on the three-dimensional actual data set 105-I, the learned algorithm performs a calculation of how the dental prosthesis 100 should look. The user can then optimize the esthetics. The learned algorithm can automatically determine the functionally important areas of the dental prosthesis 100, such as a functional margin or esthetic lines. The functional margin is the part of the prosthesis margin that is determined by the functional movements (opening of the mouth, swallowing, pursing of the lips) of the surrounding soft tissues.

For the user, there is a time and cost saving, as no model impressions have to be sent. Instead, the dental prosthesis can be designed directly on the computer. This means that the dental prosthesis can be produced with fewer steps and in a shorter time. For example, the method implements a generative network to propose a prosthesis.

In addition, the data in the input data set 105-1 may additionally comprise the following information:
color information, such as of the soft tissue or the residual dentition;
information about the surrounding soft tissue, such as a thickness or elasticity of cheeks and lips;
information on a residual dentition, implants or implant planning;
information on a digital volume tomography (DVT) or image information;
information on the patient's health status, such as a degree of atrophy or a quality of the mucosa;
information about an age, gender, skin color, or type, such as Caucasian, Asian;
movement information of the lower jaw; and
information about a transfer bow, such as photos or videos with the transfer bow in the intraoral space.

The method can then calculate a tooth set-up or tooth set in addition to determining the functional surface 101. In the case of a partial prosthesis, the method can calculate the design of the prosthesis, anchorage variants, and/or construction options for the partial prosthesis.

Once the functional surface 101 has been determined, the dental prosthesis 100 can be produced from a blank using a milling process, for example. The anatomical and esthetic information required to produce the dental prosthesis can be retrieved automatically from a database.

Defined auxiliary prostheses can also be used, which the patient can insert temporarily. These can be made in different sizes and shapes or with interchangeable elements so that they can be customized. A photo is then taken with each auxiliary prosthesis or a scan and speech test are performed. Then the optimal variant in terms of aesthetics and pronunciation is calculated. In addition, the dentist can temporarily draw esthetic lines on these auxiliary prostheses, which serve as further input for the system.

For example, the method can be implemented by the following code:
path_to_successful_patient_cases=“C: \Users\Desktop\SuccessfulPatientCases” dataset_input=initialize_empty_dataset( ) dataset_expected_output=initialize_empty_dataset( ) n=0 for individual_patient_case in get_data(path_to_successful_patient_cases): patient_n.input.intraoral_scan_data=load(individual_patient_case.scandata) patient_n.output.functional_margin=load(individual_patient_case.functional_margin)
if individual_patient_case.intraoral_images.exists( ): patient_n.input.intraoral_images=load(individual_patient_case.intraoral_images)
if individual_patient_case.face_images.exists( ): patient_n.input.face_images=load(individual_patient_case.face_images)
if individual_patient_case.face_scan.exists( ): patient_n.input.face_scan=load(individual_patient_case.face_scan)
if individual_patient_case.landmarks.exists( ): patient_n.input.landmarks=load(individual_patient_case.landmarks)
if individual_patient_case.denture_design.exists( ): patient_n.output.denture_design=load(individual_patient_case.denture_design)
if individual_patient_case.denture_design_parameter.exists( ): patient_n.output.denture_design_parameter=load(individual_patient_case.denture_design_parameter)
dataset_input.add(patient_n.input) dataset_expected_output.add(patient_n.output) n+=1
dataset_train=make_dataset(dataset_input, dataset_expected_output) model=load_model(model_type=“GAN”) compile_options=load_compile_options(model_type=“GAN”)
model.compile (compile_options) model.train(dataset_train, epochs=50) model.save(‘denture_designer_model’)
#use model model=load(‘denture_designer_model’)
patient_who_needs_a_denture.intraoral_scan_data=load(patient_who_needs_a_denture.scandata)
if patient_who_needs_a_denture.intraoral_images.exists( ) patient_who_needs_a_denture.intraoral_images=load(patient_who_needs_a_denture_case.intraoral_images)
if patient_who_needs_a_denture.face_images.exists( ): patient_who_needs_a_denture.face_images=load(patient_who_needs_a_denture_case.face_images)
if patient_who_needs_a_denture.face_scan.exists( ): patient_who_needs_a_denture.face_scan=load(patient_who_needs_a_denture_case.face_scan)
if patient_who_needs_a_denture.landmarks.exists( ): patient_who_needs_a_denture.landmarks=load(patient_who_needs_a_denture_case.landmarks)
functional_margin, denture_design, denture_design_parameter=model.evaluate(patient_who_needs_a_denture)

All of the features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by the features of the subject matter can be a method step of a method.

The scope of protection of the present invention is given by the claims and is not limited by the features explained in the description or shown in the Figures.

### REFERENCE LIST

100 Dental prosthesis
101 Functional surface
103 Intraoral space
105 Data set
107 Intraoral scanner
109 Algorithm
200 System

1. A method of determining the three-dimensional shape of a functional surface of a dental prosthesis, comprising the steps of:
detecting an intraoral space to generate a three-dimensional actual data set; and
inputting the three-dimensional data set into a learned algorithm for generating a three-dimensional nominal data set for a functional surface of the dental prosthesis.

2. The method according to claim 1, wherein the learned algorithm comprises an artificial neural network.

3. The method according to claim 2, wherein the artificial neural network comprises multiple layers.

4. The method according to claim 3, wherein the artificial neural network comprises a convolution layer, a pooling layer, and/or a dense layer.

5. The method according to claim 1,
wherein the intraoral space is scanned with a scanner.

6. The method according to claim 1,
wherein the functional surface is a contact surface of the dental prosthesis to the soft tissue.

7. The method according to claim 1,
wherein the three-dimensional actual data set comprises additional data on a soft tissue elasticity.

8. The method according to claim 1,
wherein the three-dimensional actual data set comprises additional spatial data on an upper jaw, a lower jaw and/or a face.

9. The method according to claim 1,
wherein the learned algorithm calculates a functional margin of the dental prosthesis.

10. The method according to claim 1,
wherein the learned algorithm calculates a tooth set-up or tooth set.

11. The method according to claim 1,
wherein the dental prosthesis is produced with the generated functional surface.

12. A system for determining a three-dimensional shape of a functional surface of a dental prosthesis; comprising:
an intraoral scanner for scanning an intraoral space to generate a three-dimensional actual data set; and
a learned algorithm for inputting the three-dimensional data set and outputting a three-dimensional nominal data set for a functional surface of the dental prosthesis.

13. The system according to claim 12,
wherein the learned algorithm comprises an artificial neural network.

14. The system according to claim 12,
wherein the system comprises a production device for producing the dental prosthesis with the generated functional surface.

15. A computer program product comprising program code which is stored on a non-transitory machine-readable medium, the machine-readable medium comprising computer instructions executable by a processor, which computer instructions cause the processor to perform the method according to claim 1.
